(19) United States
(12) Patent Application Publication
(10) Pub. No.: US 2025/0194984 A1
(43) Pub. Date: Jun. 19, 2025

(54) MULTI-LOOP MAPPING CATHETER

(71) Applicant: Boston Scientific Scimed, Inc., Maple Grove, MN (US)

(72) Inventors: Jackie Leung, Richmond Hill (CA); Lauren Koon, Mississauga (CA)

(21) Appl. No.: 18/972,675

(22) Filed: Dec. 6, 2024

Related U.S. Application Data
(60) Provisional application No. 63/610,804, filed on Dec. 15, 2023.

(51) Int. Cl. A61B 5/287; A61B 5/00; A61B 5/06; A61B 18/00; A61B 18/12; A61B 18/14

(52) U.S. Cl. CPC A61B 5/287; A61B 5/065; A61B 5/6859; A61B 18/1206; A61B 18/1492; A61B 2018/0016; A61B 2018/00214; A61B 2018/00351; A61B 2018/00577; A61B 2018/00839; A61B 2560/0468; A61B 2562/043

(57) ABSTRACT

A catheter to detect a plurality of physiological signal from within a patient's heart is disclosed. The catheter includes an elongated shaft having a distal region. A location sensor is coupled to the distal region. First and second sensing electrode arrays with electrodes are coupled to the distal region. The first sensing electrode array includes a first spline formed as first non-planar loop in an expanded configuration wherein first and second ends are coupled to the distal region of the shaft and a first intermediate portion extends from the distal region. The second sensing electrode array includes a second spline formed as second non-planar loop in the expanded configuration wherein third and fourth ends are coupled to the distal region of the shaft and a second intermediate portion extends from the distal region. The first intermediate portion does not contact the second intermediate portion in the expanded configuration.

### CROSS REFERENCE TO RELATED APPLICATIONS

This application claims priority to U.S. Provisional Application No. 63/610,804 entitled “MULTI-LOOP MAPPING CATHETER,” filed Dec. 15, 2023, which is hereby incorporated by reference in its entirety.

### TECHNICAL FIELD

The present disclosure relates to medical systems and methods for mapping an anatomical space of the body. More specifically, the present disclosure relates to high density mapping catheters and systems and methods for cardiac, electroanatomical mapping such as in minimally invasive electrophysiological procedures.

### BACKGROUND

Electrophysiological procedures, which include catheter ablation to treat a variety of heart conditions such as supraventricular and ventricular arrhythmia, involve a visualization of the heart and heart activity. Electroanatomical mapping is a visualization technique that allows a clinician to accurately determine the location of an arrhythmia, define cardiac geometry in three dimensions, delineate areas of anatomic interest, and permits imaging of the catheter assembly for positioning and manipulation. For instance, electroanatomical mapping involves the mapping of electrical activity in the heart based on cardiac signals, such as at various locations on the endocardium surface, to identify the site of origin of the arrhythmia followed by a targeted ablation of the site.

To perform such cardiac mapping, a catheter with an electrode assembly at a distal tip of the catheter can be inserted into the patient's heart chamber. In some examples of mapping, physiological signals from electrical activity of the heart are acquired with electrodes after the tip is in stable and steady contact with the endocardium surface of a particular heart chamber. Alternatively, or additionally, signals can be detected by non-contact electrodes along with information on chamber anatomy and relative electrode location to provide physiological information regarding the endocardium of the heart chamber. The locations of the physiological signals are determined, such as via location sensors on or near the electrode assembly. Location and electrical activity are measured, such as sequentially on a point-by-point basis in some examples, at about fifty to several hundred points on the internal surface of the heart to construct an electroanatomical map of the heart. The generated electroanatomical map can serve several purposes, such as the basis to decide on a therapeutic course of action like tissue ablation, which can be applied to alter the propagation of electrical activity in the heart and to restore normal heart rhythm.

### SUMMARY

In Example 1, a catheter to detect a plurality of physiological signal from within a patient's heart, the catheter comprising: an elongated shaft having a proximal region and a distal region, the elongated shaft defining a longitudinal axis; a location sensor coupled to the distal region and configured to generate a plurality of location signals representative of a location of the catheter within the patient's heart; and a plurality of sensing electrode arrays coupled to the distal region, the plurality of sensing electrode arrays having a collapsed configuration and an expanded configuration, the plurality of sensing electrode arrays including a first sensing electrode array and a second sensing electrode array. The first sensing electrode array, comprising: a first spline having a first end, an opposite, second end, and a first intermediate portion between the first end and the second end, the first spline formed as first non-planar loop in the expanded configuration wherein the first and second ends are coupled to the distal region of the shaft and first intermediate portion extends from the distal region; and a first plurality of electrodes disposed on the first intermediate portion. The second sensing electrode array, comprising: a second spline having a third end, an opposite, fourth end, and a second intermediate portion between the third end and the fourth end, the second spline formed as second non-planar loop in the expanded configuration wherein the third and fourth ends are coupled to the distal region of the shaft and second intermediate portion extends from the distal region; and a second plurality of electrodes disposed on the second intermediate portion; wherein the first intermediate portion does not contact the second intermediate portion in the expanded configuration.
In Example 2, the catheter of Example 1, wherein the plurality of sensing electrode arrays includes two sensing electrode arrays.
In Example 3, the catheter of any of Examples 1 and 2, wherein the plurality of sensing electrode arrays are deformable from the expanded configuration.
In Example 4, the catheter of Example 3, wherein the first non-planar loop includes first bends in the first spline and the second non-planar loop includes second bends in the second spline, the first and second bends configured to flex when deformed from the expanded configuration.
In Example 5, the catheter of Example 4, wherein the first bends include a first distal bends and first proximal bends, and the second bends include second distal bends and second proximal bends.
In Example 6, the catheter of any of Examples 1-5, wherein the first non-planar loop includes a first distal planar loop section in a first plane and the second non-planar loop includes a second distal planar loop section in a second plane in the expanded configuration.
In Example 7, the catheter of Example 6, wherein the first plane lies in the second plane in the expanded configuration, the first and second planes perpendicular to the longitudinal axis.
In Example 8, the catheter of Example 6, wherein the first planar loop section and second planar loop section are deformable from the expanded configuration such that the first plane lies in the second plane, the first and second planes perpendicular to the longitudinal axis.
In Example 9, the catheter of Example 6, wherein the first plane is parallel to the second plane in the expanded configuration, the first and second planes parallel to a reference plane including the longitudinal axis.
In Example 10, the catheter of Example 9, wherein the first non-planar loop includes a first medial planar loop section in a third plane and the second non-planar loop includes a second medial loop section in the third plane, the third plane perpendicular to the longitudinal axis in the expanded configuration.
In Example 11, the catheter of any of Examples 1-6, wherein the first non-planar loop extends from a first side to a second side of a reference plane including the axis, and the second non-planar loop extends from a second side to a first side of the reference plane in the expanded configuration.
In Example 12, the catheter of any of Examples 1-11, wherein the location sensor is disposed in the shaft.
In Example 13, the catheter of any of Examples 1-12, wherein the plurality of sensing arrays are configured to tilt off axis from the shaft.
In Example 14, the catheter of any of Examples 1-13 included in an electrophysiological system having an electroanatomical mapping system and configured to map the patient's heart with the sensing electrode arrays.
In Example 15, the catheter of Example 14, wherein the electrophysiological system is configured to ablate tissue in the heart.
In Example 16, a catheter to detect a plurality of physiological signal from within a patient's heart, the catheter comprising: an elongated shaft having a proximal region and a distal region, the elongated shaft defining a longitudinal axis; a location sensor coupled to the distal region and configured to generate a plurality of location signals representative of a location of the catheter within the patient's heart; and a plurality of sensing electrode arrays coupled to the distal region, the plurality of sensing electrode arrays having a collapsed configuration and an expanded configuration, the plurality of sensing electrode arrays including a first sensing electrode array and a second sensing electrode array. The first sensing electrode array, comprising: a first spline having a first end, an opposite, second end, and a first intermediate portion between the first end and the second end, the first spline formed as first non-planar loop in the expanded configuration wherein the first and second ends are coupled to the distal region of the shaft and first intermediate portion extends from the distal region; and a first plurality of electrodes disposed on the first intermediate portion. The second sensing electrode array, comprising: a second spline having a third end, an opposite, fourth end, and a second intermediate portion between the third end and the fourth end, the second spline formed as second non-planar loop in the expanded configuration wherein the third and fourth ends are coupled to the distal region of the shaft and second intermediate portion extends from the distal region; and a second plurality of electrodes disposed on the second intermediate portion; wherein the first intermediate portion does not contact the second intermediate portion in the expanded configuration.
In Example 17, the catheter of Example 16, wherein the plurality of sensing electrode arrays includes two sensing electrode arrays.
In Example 18, the catheter of Example 16, wherein the plurality of sensing electrode arrays are deformable from the expanded configuration.
In Example 19, the catheter of Example 18, wherein the first non-planar loop includes first bends in the first spline and the second non-planar loop includes second bends in the second spline, the first and second bends configured to flex when deformed from the expanded configuration.
In Example 20, the catheter of Example 19, wherein the first bends include a first distal bends and first proximal bends, and the second bends include second distal bends and second proximal bends.
In Example 21, the catheter of Example 16, wherein the first non-planar loop includes a first distal planar loop section in a first plane and the second non-planar loop includes a second distal planar loop section in a second plane in the expanded configuration.
In Example 22, the catheter of Example 21, wherein the first plane lies in the second plane in the expanded configuration, the first and second planes perpendicular to the longitudinal axis.
In Example 23, the catheter of Example 21, wherein the first planar loop section and second planar loop section are deflectable from an expanded configuration such that the first plane lies in the second plane, the first and second planes perpendicular to the longitudinal axis.
In Example 24, the catheter of Example 21, wherein the first plane is parallel to the second plane in the expanded configuration, the first and second planes parallel to a reference plane including the longitudinal axis.
In Example 25, the catheter of Example 24, wherein the first non-planar loop includes a first medial planar loop section in a third plane and the second non-planar loop includes a second medial loop section in the third plane, the third plane perpendicular to the longitudinal axis in the expanded configuration.
In Example 26, the catheter of Example 16, wherein the first non-planar loop extends from a first side to a second side of a reference plane including the axis, and the second non-planar loop extends from a second side to a first side of the reference plane in the expanded configuration.
In Example 27, the catheter of Example 16, wherein the location sensor is disposed in the shaft.
In Example 28, the catheter of Example 16, wherein the plurality of sensing arrays are configured to tilt off axis from the shaft.
In Example 29, an electrophysiology system, comprising: a catheter to detect a plurality of physiological signal from within a patient's heart, the catheter comprising: an elongated shaft having a proximal region and a distal region, the elongated shaft defining a longitudinal axis; a location sensor coupled to the distal region and configured to generate a plurality of location signals representative of a location of the catheter within the patient's heart; and a plurality of sensing electrode arrays coupled to the distal region, the plurality of sensing electrode arrays having a collapsed configuration and an expanded configuration, the plurality of sensing electrode arrays including a first sensing electrode array and a second sensing electrode array; the first sensing electrode array, comprising: a first spline having a first end, an opposite, second end, and a first intermediate portion between the first end and the second end, the first spline formed as first non-planar loop in the expanded configuration wherein the first and second ends are coupled to the distal region of the shaft and first intermediate portion extends from the distal region; and a first plurality of electrodes disposed on the first intermediate portion; the second sensing electrode array, comprising: a second spline having a third end, an opposite, fourth end, and a second intermediate portion between the third end and the fourth end, the second spline formed as second non-planar loop in the expanded configuration wherein the third and fourth ends are coupled to the distal region of the shaft and second intermediate portion extends from the distal region; and a second plurality of electrodes disposed on the second intermediate portion; wherein the first intermediate portion does not contact the second intermediate portion in the expanded configuration; and an electroanatomical mapping system coupled to the catheter and configured to receive signals representative of the detected physiological signals from the plurality of sensing electrode arrays and map the patient's heart.
In Example 30, the electrophysiological system of Example 29, and further comprising a console coupled to the catheter, the console configured to provide ablation energy to the catheter.
In Example 31, a catheter to detect a plurality of physiological signal from within a patient's heart, the catheter comprising: an elongated shaft having a proximal region and a distal region, the elongated shaft defining a longitudinal axis; a location sensor coupled to the distal region and configured to generate a plurality of location signals representative of a location of the catheter within the patient's heart; and a pair of sensing electrode arrays coupled to the distal region, the pair of sensing electrode arrays having a collapsed configuration and an expanded configuration, the pair of sensing electrode arrays including a first sensing electrode array and a second sensing electrode array. The first sensing electrode array, comprising: a first spline having a first end, an opposite, second end, and a first intermediate portion between the first end and the second end, the first spline formed as first non-planar loop in the expanded configuration wherein the first and second ends are coupled to the distal region of the shaft and first intermediate portion extends from the distal region; and a first plurality of electrodes disposed on the first intermediate portion. The second sensing electrode array, comprising: a second spline having a third end, an opposite, fourth end, and a second intermediate portion between the third end and the fourth end, the second spline formed as second non-planar loop in the expanded configuration wherein the third and fourth ends are coupled to the distal region of the shaft and second intermediate portion extends from the distal region, the second spline opposite the first spline; and a second plurality of electrodes disposed on the second intermediate portion; wherein the first intermediate portion does not contact the second intermediate portion in the expanded configuration.
In Example 32, the catheter of Example 31, wherein the first non-planar loop includes a first distal planar loop section in a first plane and the second non-planar loop includes a second distal planar loop section in a second plane in the expanded configuration.
In Example 33, the catheter of Example 32, wherein the first plane intersects the second plane in the expanded configuration.
In Example 34, the catheter of Example 33, wherein the first planar loop section and second planar loop section are deformable from the expanded configuration such that the first plane lies in the second plane, the first and second planes perpendicular to the longitudinal axis.
In Example 35, the catheter of Example 33, wherein the first non-planar loop extends from a first side to a second side of a reference plane including the axis, and the second non-planar loop extends from a second side to a first side of the reference plane in the expanded configuration.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system.

FIG. 2 is a perspective view of a distal portion of an embodiment of a dual loop catheter for use in the example system of FIG. 1.

FIG. 3 is a perspective view of a distal portion of another embodiment of a dual loop catheter for use in the example system of FIG. 1.

FIG. 4 is a perspective view of a distal portion of another embodiment of a dual loop catheter for use in the example system of FIG. 1.

FIG. 5 is a perspective view of a distal portion of another embodiment of a dual loop catheter for use in the example system of FIG. 1.

FIG. 6 is a perspective view of a distal portion of another embodiment of a dual loop catheter for use in the example system of FIG. 1.

FIGS. 7A and 7B are side views of the embodiment of the dual loop catheter of FIG. 6 while in operation in a heart chamber.

FIGS. 8A and 8B are perspective views of the embodiment of the dual loop catheter of FIG. 6 while in operation in a pulmonary vein of the heart.

FIGS. 9A-9F are side views of the embodiment of the dual loop catheter of FIG. 6 as the catheter transitions from a collapsed state to an expanded state.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

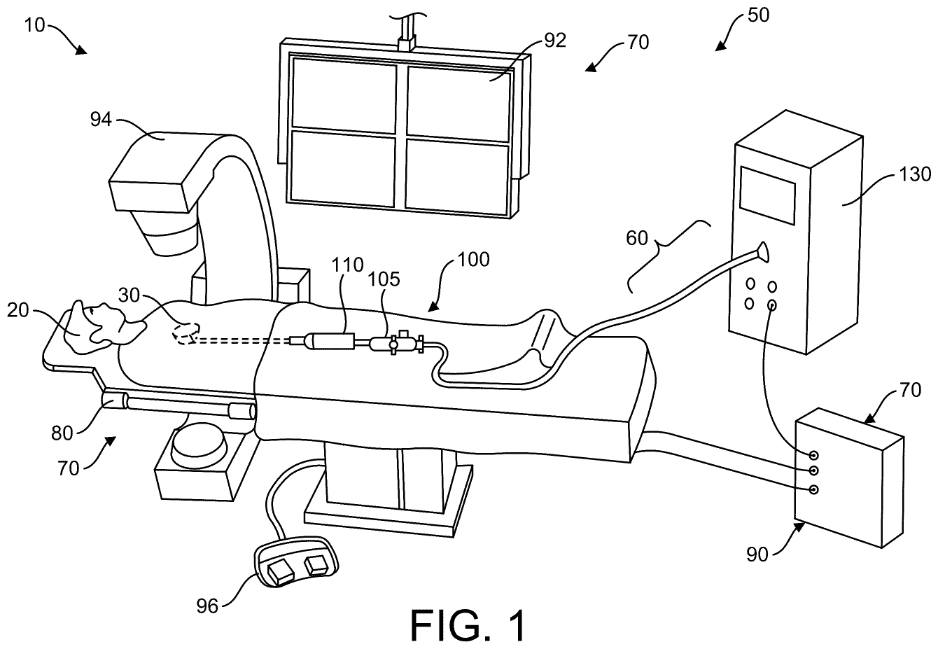

FIG. 1 illustrates an example clinical setting 10 for treating a patient 20, which can include mapping a heart 30 of the patient 20, using an electrophysiology system 50, in accordance with the disclosure. The electrophysiology system 50 includes a catheter system 60 and an electroanatomical mapping (EAM) system 70. The example catheter system 60 includes a catheter 105 to obtain physiological (electrical) signals of the heart, an introducer sheath 110, and can include a console 130. In some examples, the console can provide ablation energy such as in the case of the catheter 105 configured as a mapping and ablation catheter. Additionally, the catheter system 60 includes various connecting elements, such as cables, that operably connect the components of the catheter system 60 to one another and to the components of the EAM system 70. In general, the EAM mapping system 70 includes a localization field generator 80, a mapping and navigation controller 90, and a display 92. The EAM mapping system 70 is configured to receive the electrical signals obtained from the heart, such as in the case of a mapping catheter. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The introducer sheath 110 is operable to provide a delivery conduit through which the catheter 105 can be deployed to the specific target sites within the patient's heart 30. Access to the patient's heart can be obtained through a vessel, such as a peripheral artery or vein. Once access to the vessel is obtained, the catheter 105 can be navigated to within the patient's heart, such as within a chamber of the heart.

The example catheter 105 includes an elongated catheter shaft and distal end region configured to be deployed proximate target tissue, such as within a chamber of the patient's heart. The shaft can extend from an access point in the patient to the target tissue and generally defines a longitudinal axis of the catheter 105. The distal end region may include an electrode deployment mechanism coupled to the shaft. The electrode deployment mechanism includes a plurality of electrode assemblies, each electrode assembly having one or more electrodes, to obtain electrical signals from the heart. For example, the electrode assemblies can include a plurality of spaced-apart electrodes or multiple spaced-apart sets or groups of spaced-apart electrodes. For instance, the electrode deployment mechanism includes a plurality of splines, and at least a some of the electrodes are disposed on the splines.

The catheter 105 is capable of being formed into a plurality of configurations. For example, when the distal end region of the catheter 105 is within a sheath as a catheter assembly, such as to travel to the patient to the chamber of the heart, the electrode deployment mechanism and electrode assembly are in a collapsed state to fit within the sheath. Once the catheter has reached the destination in the chamber of the heart, for example, the sheath is retracted from the distal region of the catheter 105 (or the shaft catheter is extended past the sheath), and the electrode deployment mechanism and electrode assembly can be arranged in an expanded configuration. In the expanded configuration, the forces on the electrode deployment mechanism and electrode assembly are not sufficient to deform the electrode deployment mechanism and electrode assembly. In some embodiments, a flexible electrode deployment mechanism and electrode assembly can assume a conformed state, such as the electrode deployment mechanism and electrode assembly are subjected to forces that deform the expanded state, such as in the example of the flexible electrode deployment mechanism and electrode assembly in an expanded state and then pressed against tissue wall to deform the shape of the electrode deployment mechanism and electrode assembly.

In one example, the plurality of electrodes can be formed of a conductive, solid-surface, biocompatible material and are spaced-apart across insulators. Each of the plurality of electrodes is electrically coupled to a corresponding elongated lead conductor that extend along the shaft to a catheter proximal end. In one example, each electrode of the spaced-apart electrodes corresponds with a separate, single lead conductor. Other configurations are contemplated. The plurality of lead conductors can be insulated from one another within an insulating sheath along the catheter shaft, such as with an insulating polymer sheath. The lead conductors can be electrically coupled to plug in the proximal region of the catheter 105, such as a plug configured to be mechanically and electrically coupled to the console 130, for example, either directly or via intermediary electrical conductors such as cabling.

In some embodiments, the electrode assemblies and associated electrodes are configured for, among other things, sensing cardiac electrical signals, ablation, localization of the electrode assembly within the patient anatomy such as via the EAM system 70, signal reference, and to determine proximity to target tissue within the anatomy. In some embodiments, the catheter 105 is configured for cardiac mapping, and the electrodes are sensing, or mapping, electrodes configured to be used to collect physiological (electrical) signals to be used to generate electroanatomical maps. In some embodiments, the catheter 105 can be a mapping and ablation catheter, and the electrodes can include ablation electrodes that are configured to deliver ablation electric field energy and sensing electrodes as well as mapping electrodes, for mapping purposes. The ablation electrodes in embodiments of an electroporation catheter are configured to receive pulsed electrical signals or waveforms from the console 130 and create pulsed electric fields sufficient to ablate target tissue via irreversible electroporation. The mapping electrodes in the electrode assembly can be electrically coupled to a one or more lead conductors that extends the length of the shaft that are configured to carry an electrical signal received at the mapping electrode. In some examples, an electrode in the electrode assembly can be configured to only perform an ablation or the electrode in the electrode assembly can be configured to only perform mapping. In some examples, an electrode can operate as an ablation electrode in an ablation mode of the electrophysiology system 50 and as a mapping electrode in a mapping mode of the system 50. Some examples of mapping and ablation catheters are smaller in profile or in the volume of the electrode assembly than catheters that just perform mapping, and clinicians can map a given location within the heart with fewer passes across the chamber with mapping catheters than with mapping and ablation catheters.

Mapping electrodes on the catheter 105 can measure physiological (electrical) signals and generate electrical output signals that can be processed by the mapping and navigation controller 90 to generate an electroanatomical map. In some configurations, the mapping electrodes are configured to be used to collect physiological electrical signals to be used to generate via the operably coupled EAM system 70, and display via the operably coupled display 92, detailed three-dimensional geometric anatomical maps or representations of the cardiac chambers as well as electro-anatomical maps in which cardiac electrical activity of interest is superimposed on the geometric anatomical maps. In some instances, electroanatomical maps are generated before ablation for determining the electrical activity of the cardiac tissue within a chamber of interest. In some instances, electroanatomical maps are generated after ablation in verifying the desired change in electrical activity of the ablated tissue and the chamber. The mapping electrodes may also be used to determine the position of the catheter 105 in three-dimensional space within the body. For example, when the operator moves the distal end of the catheter 105 within a cardiac chamber of interest, the boundaries of catheter movement can be used by the mapping and navigation controller 90 to form the anatomical map of the chamber. The electroanatomical map may be used to facilitate navigation of the catheter 105 without the use of ionizing radiation such as with fluoroscopy, and for tagging locations of ablations as they are completed to guide spacing of ablations and aid the clinician in ablating the anatomy of interest.

The EAM system 70 is configured to generate the electroanatomical map for display on the display 92. The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the RHYTHMIA™ HDx mapping system marketed by Boston Scientific Corporation. Also, the mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the field generator 80, to define a localization volume about the heart 30, and a location sensor or sensing element on a tracked device, such as sensors on the catheter 105, generate an output that can be processed by the mapping and navigation controller 90 to track the location of the sensor, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices, such as in addition to or instead of magnetic tracking. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the RHYTHMIA HDx™ mapping system.

Regardless of the tracking methodology employed, the EAM system 70 utilizes the location information for the various tracked devices, along with cardiac electrical activity acquired by, for example, the catheter 105 or another catheter or probe equipped with sensing electrodes, to generate, and display via the display 92, detailed three-dimensional geometric anatomical maps or representations of the heart tissue and voids such as cardiac chambers as well as electroanatomical maps in which cardiac electrical activity of interest is superimposed on the geometric anatomical maps.

Each cardiac physiological (electrical) signal can include several intracardiac electrograms (EGMs) sensed within a patient's heart and may include any number of features that may be ascertained by aspects of the system 50. Examples of cardiac physiological signal features include activation times, activations, activation waveforms, filtered activation waveforms, minimum voltage values, maximum voltages values, maximum negative time-derivatives of voltages, instantaneous potentials, voltage amplitudes, dominant frequencies, and peak-to-peak voltages. A cardiac physiological signal feature can refer to one or more features extracted from one or more cardiac physiological signals, derived from one or more features that are extracted from one or more cardiac physiological signals. Additionally, a representation, on a cardiac or a surface map, of a cardiac physiological signal feature may represent one or more cardiac physiological signal features, an interpolation of several cardiac physiological signal features. Each cardiac physiological signal also can be associated with a set of respective position coordinates that corresponds to the location at which the cardiac physiological signal was sensed. Each of the respective position coordinates for the sensed cardiac physiological signals can include three-dimensional Cartesian coordinates, polar coordinates, or another coordinate system. The cardiac physiological signals may be sensed on the cardiac surfaces, and the respective position coordinates can be on the endocardial surface, epicardial surface, in the mid-myocardium of the patient's heart, or in a vicinity.

During a signal-acquisition stage of a cardiac mapping procedure, the catheter 105 is displaced to multiple locations within the heart chamber into which the catheter 105 is inserted. At each location to which the catheter 105 is moved, the electrodes and sensors acquire physiological signals resulting from the electrical activity in the heart along with positional, or spatial, information of the catheter 105. The spatial information is used in building a three-dimensional grid of the anatomy during mapping. To perform a mapping procedure and reconstruct physiological information on the endocardium surface, the EAM system 70 may align a coordinate system of the catheter 105 with the endocardium surface's coordinate system, or vice versa. Alternatively, or additionally, the grid may be used to capture EGMs, and select mapping values based on statistical distributions associated with nodes of the grid. The EAM system 70 also can perform post-processing operations on the physiological information to extract and display useful features of the information to the operator of the system 50.

In generating an example electroanatomical map, a data stream including multiple signals, such as signals received from the mapping electrodes of the catheter 105, is input into the EAM system 70. During the automated electroanatomical mapping process, the data stream provides a collection of physiological and location signals that serve as an input to the mapping process. The signals may be collected directly by the mapping system, obtained from another system using an analog or digital interface, or both. The data stream 302 can include signals such as unipolar and/or bipolar intracardiac EGMs, surface electrocardiograms (ECGs), electrode location information originating from one or more of a variety of methodologies, tissue proximity information, catheter force information, catheter to tissue contact information, catheter temperature, acoustic information, catheter electrical coupling information, catheter deployment shape information, electrode properties, respiration phase, blood pressure, and other physiological information. For the generation of specific types of maps, one or more signals may be used as one or more references to trigger and align the data stream relative to a cycle or clock, which can be used to create beat datasets. Beat metrics can be determined from the beat datasets. A beat acceptance process can be applied to determine which beat datasets will make up a map dataset. The map dataset may be stored in association with a three-dimensional grid that is dynamically generated during data acquisition.

Surface geometry data of the cardiac surface is generated, such as generated concurrently, during the data acquisition process using acceptance metrics employing a surface geometry construction process. This process constructs surface geometry using data such as electrode locations and catheter shape contained in the data stream. Additionally, or alternatively, previously collected surface geometry of the cardiac surface can be used as an input to surface geometry data. Previously collected geometry may have been collected using a different map dataset or using a different modality such as computerized tomography (CT), magnetic resonance imaging (MRI), ultrasound, or rotational angiography and registered to the catheter locating system. A surface map generation process is employed to generate surface map data from the map dataset and surface geometry data.

The depiction of the electrophysiology system 50 shown in FIG. 1 is intended for illustration or a general overview of the various components of the system 50 and is not intended to imply that the disclosure is limited to any set of components or arrangement of the components. For example, additional hardware components, such as breakout boxes or workstations, can be included in the electrophysiology system 50.

High density mapping catheters for use in the electrophysiology system 50, i.e., catheters configured to detect a plurality of physiological signals from within a patient's heart, generally are available in one of three designs. A first design is a basket or globe configuration that includes a set of splines or struts coupled together at a proximal end and a distal end and formed in a selected shape such as a sphere or ellipsoid. Basket or globe configurations provide for a full range of sensing capability, can be readily coupled with ablation electrodes, and are often configured with many features. Accordingly, basket or globe configurations can include complex designs that are expensive to manufacture. Another design is a spline catheter having set of splines radially extending from an end of a shaft like spokes of wheel from a hub. Spline catheters typically include a less complicated design, but the flexible splines can assume an unpredictable shape that is prone to tangling with structures of the anatomy. Additionally, the variable distance between the deflectable splines can generate maps with a fair amount of noise. Still another design is a paddle catheter having a set of electrodes on struts configured in two-dimensions, such as a plane in the axis of the shaft. In many examples, the tracking sensors are relatively far from the sensing electrodes, which can lead to errors in detecting the positions of the physiological signals. Further, the electrodes of paddle catheters are perhaps more suited to detect physiological signals to the side of the shaft rather than in front of the catheter unless the paddle can be bent about ninety degrees against the tissue.

Embodiments of the present disclosure provide for high density mapping catheters that configured to sense physiological signals and, in some examples, ablate tissue is a design that can be easier to manufacture and less expensive than many basket catheters. The embodiments include a plurality of electrode assemblies formed as memory shaped loops for stability. In some examples, the loops include sets of parallel electrodes relatively close to the tracking sensors to generate high fidelity maps of relatively accurate physiological positions. For instance, two loops can provide for four splines of parallel electrodes. The atraumatic design can assume many configurations and are suited for detecting physiological signals in front of the catheter as well as to the side of the catheter. Further, the electrode assemblies are readily steerable and easy to use.

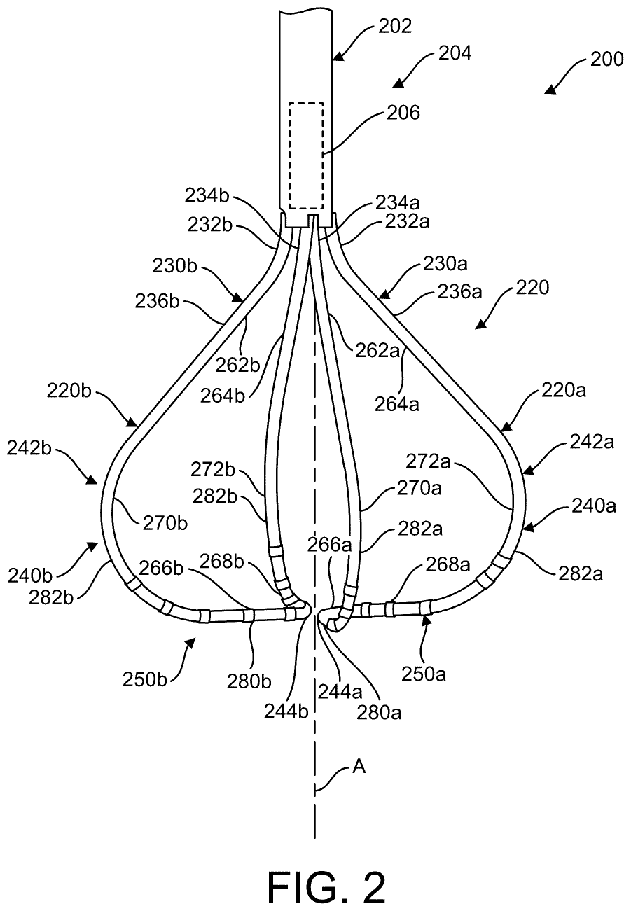

FIG. 2 is a partial perspective illustration of an embodiment of a catheter 200 configured to detect a plurality of physiological signals from within a patient's heart. The catheter 200 corresponds with the catheter 105 described with respect to FIG. 1 and can be used in the electrophysiology system 50. For example, catheter 200 can be configured as a mapping catheter in some embodiments and as a mapping and ablation catheter in some embodiments. The catheter 200 has an elongated shaft 202 having a proximal region and a shaft distal region 204 configured to be deployed proximate target tissue, such as within a chamber of the patient's heart. The elongated shaft 202 defines a longitudinal axis A, which is presented as a line passing through a centroid of a cross section of the shaft 202. In the illustrated embodiment, the catheter 202 includes a location sensor 206 (shown in phantom) coupled to the distal region 204 can be configured to generate a plurality of location signals representative of a location of the catheter 200 within the patient's heart. A plurality of sensing electrode assemblies 220 are coupled to the distal end region 204. The plurality of sensing electrode assemblies 220 can include two or more sensing electrode assemblies 220. For example, the plurality of sensing electrode assemblies 220 can include at least a first sensing electrode assembly 220a and a second sensing electrode assembly 220b.

In embodiments, the elongate shaft 202 is formed of a biocompatible material that provide sufficient sturdiness and flexibility to allow the shaft 202 to be navigated through the vasculature of a patient and reach the treatment site, such as a chamber of the heart. In some embodiments, the shaft 202 is formed of multiple different materials to provide the catheter 200 with more flexibility at the distal region 204 than a proximal region. Further, the shaft 202 can included a tubular woven member to provide torsional stiffness and bending flexibility. The shaft 202 can include various markers for use with a visualization system, such as radiopaque or echogenic markers, or EAM electrodes to facilitate visualization and location. The catheter shaft 202 can also accommodate pull wires or other mechanisms to direct, bend or tilt the electrode assemblies 220 to the treatment site. With pull wires or other mechanisms, the angle of the electrode assemblies 220 with respect to the shaft 202 can be selected and varied, and the relationship of the electrode assemblies 220 to the shaft 202 can be adjusted. The distal region 204 can include sensors such as one or more tracking, or location sensors 206. For example, the location sensor 206 can include a magnetic sensor for tracking the location of the catheter 200 within a magnetic location field or an EAM electrode for tracking the location of the catheter 200 within an electric location field. In one embodiment, the location sensor is a magnet location sensor disposed within the shaft 202. In other embodiments, the location sensor 206 can be disposed on the electrode assemblies 220. The distal region 204 can also include force sensors and additional elements such as an irrigation element. In some embodiments, a stem is included extending from the elongate shaft 202, and the stem is configured to include irrigation elements and sensors and other components.

The sensing electrode assemblies 220 includes a collapsed configuration and an expanded configuration (as illustrated in FIG. 2). In embodiments, the sensing electrode assemblies 220 are arranged in the collapsed configuration to fit within a catheter sheath. For example, the catheter 200 can assume the collapsed configuration to travel through a patient's vasculature, or within an opening in tissue such as a puncture in the atrial septum, to a therapy site. The sensing electrode assemblies 220 are arranged in the expanded configuration to receive physiological signals and, in embodiments, to deliver therapy. In embodiments, the sensing electrode assemblies 220 are flexible in such a manner to also receive physiological signals and deliver therapy in a conformed configuration, such as when the sensing electrode assemblies 220 in the expanded configuration are subjected to above-threshold forces that deform the shape of the sensing electrode assemblies 220 such as when pressed against a heart chamber wall. In embodiments, the sensing electrode assemblies 220 are configured to self-expand from the collapsed configuration when constrained within a delivery sheath to the pre-defined expanded configuration. For example, the sensing electrode assemblies 220 can be constructed from a material having shape-memory properties. In other examples, the controls on the catheter 200, such as on a proximal handle, can mechanically expand and collapse the sensing electrode assemblies 220.

The first sensing electrode assembly 220a includes a first spline 230a and a first plurality of electrodes 250a. The first spline 230a includes a first end 232a, an opposite, second end 234a, and a first intermediate portion 240a between the first end 232a and the second end 234a. The first spline 230a is formed as first non-planar loop 242a in the expanded configuration wherein the first and second ends 232a, 234a are coupled to the distal region 204 and first intermediate portion 240a extends from the distal region 204. The first plurality of electrodes 250a are disposed on the first intermediate portion 240a.

The second sensing electrode assembly 220b includes a second spline 230b and a second plurality of electrodes 250b. The second spline 230b includes a third end 232b, an opposite, fourth end 234b, and a second intermediate portion 240b between the third end 232b and the fourth end 234b. The second spline 230b is formed as second non-planar loop 242b in the expanded configuration wherein the third and fourth ends 232b, 234b are coupled to the distal region 204 and second intermediate portion 240b extends from the distal region 204. The second plurality of electrodes 250b are disposed on the second intermediate portion 240b. In the embodiments, the first intermediate portion 240a does not contact the second intermediate portion 240b in the expanded configuration.

In the illustrated embodiment, the first and second splines 230a, 230b are constructed from a support member 236a, 236b, respectively, and the respective set of the plurality of electrodes 250a, 250b are spaced-apart longitudinally along the associated support member 236a, 236b. In such embodiments, the sensing electrodes 250 and electrical leads are coupled directly to an outer surface of the support members 236. The sensing electrodes 250a, 250b can include pad electrode that are disposed within a periphery of the support members 232 or, in some embodiments, ring electrodes disposed around the support members 236. In some embodiments, the splines 230 are composed of a flexible circuit secured to and disposed over an outer surface of the support members. In such embodiments, the electrodes are included in the flexible circuit.

The support members 236a, 236b function, among other things, as a primary structural support of the electrode assemblies 220a, 220b, and thus primarily defines the mechanical characteristics of the electrode assemblies 220a, 220b. In embodiments, the support members 236a, 236b are formed from a superelastic material (metal or polymer) to provide desired mechanical/structural properties to the electrode assemblies 220a, 220b. In embodiments, the support members 236a, 236b are formed from a superelastic metal alloy such as a nickel-titanium alloy. Forming the support member 220 from a superelastic material such as a nickel-titanium alloy facilitates configuring the support member 220 to assume its desired unconstrained shape due to the shape memory properties of the material, while providing sufficient flexibility necessary to collapse the sensing electrode assemblies 220a, 220b within a delivery sheath. Further, the support members 236a, 236b can be constructed to include varying degrees of flexibility or rigidity along the length of the spline 230 or in selected areas of the spline 230. For example, a spline 230 can be constructed to have a relatively more rigid section or a relatively more flexible section to allow for controlled bending or shaping under stresses and forces into a conformed configuration from the expanded configuration. For instance, a spline 230 in the expanded configuration may be subjected to being pressed against a chamber wall, and the selected relatively more rigid and flexible sections will control the shape when the electrode assembly 220 is deformed. In one instance, a deformed sensing electrode assembly may include a relatively unconformable area in a relatively more rigid section of the spline 230 whereas bending and conforming will occur in a relatively more flexible section instead.

In embodiments, the splines 230a, 230b each include lateral edges having an atraumatic shape. In some embodiments, an electrically insulative material is disposed on the support members 236a, 236b such as a parylene or poly ether block amide (PEBA) coating, such as an insulative material available under the trade designations PEBAX from Arkema S.A. of Colombes, France, or VESTAMID E from Evonik Industries AG of Essen, Germany. In some embodiments, the nickel-titanium alloy is insulated with a secondary material like a polymer such as a polyimide or similar material. In embodiments that include splines 220a, 220b with a flexible circuit, the flexible circuit includes a layered construction including one or more dielectric substrate layers, and conductive traces formed on the substrate. Similar to the support member 236a, 236b, a unitary construction of the flexible circuit enhances its structural properties, for example, by minimizing joints or other discontinuities at regions subject to relatively high stresses during use.

The splines 230 are formed as non-planar loops 242 in the expanded configuration wherein the spline ends 232a, 234a, 232b, 234b are coupled to the distal region 204 and intermediate portions 240 extends from the distal region 204. Each of the loops 242a, 242b is terminated distally at an apex 244a 244b, respectively, on the associated intermediate portion 240a, 240b. The first loop 242a includes first and second proximal loop sections 262a, 264a distal the first and second spline ends 232a, 234a, respectively. The first loop 242a also includes first and second distal loop sections 266a, 268a proximal to the first apex 244a. The first loop 242a further includes first and second medial loop section 270a, 272a disposed between the first and second proximal loop sections 262a, 264a and the first and second distal loop sections 266a, 268a, respectively. The second loop 242b includes third and fourth proximal loop sections 262b, 264b distal the third and fourth spline ends 232b, 234b, respectively. The second loop 242b also include third and fourth distal loop sections 266b, 268b proximal to the second apex 244b. The second loop 242b further includes third and fourth medial loop section 270b, 272b disposed between the third and fourth proximal loop sections 262b, 264b and the third and fourth distal loop sections 266b, 268b, respectively.

In the embodiments, the first intermediate portion 240a does not contact the second intermediate portion 240b in the expanded configuration. For instance, the first and second distal loop sections 266a, 268a and first apex 244a are not coupled to the second loop 242b. And the second and third distal loop section 266b, 268b and second apex 244b are not coupled to the first loop 242a. In the embodiments, at least some of the first plurality of electrodes 250a are disposed on the first and second distal loop sections 266a, 268a, and at least some of the second plurality of electrode 250b are disposed on the third and fourth distal loop sections 266b, 268b.

The loops 242 are non-planar in the expanded configuration in that the proximal loop sections 262, 264 extend away from the axis A toward the medial sections 270, 272 in the expanded configuration; and the distal loop sections 266, 268 extend toward the axis A from the medial section 270, 272 in the expanded configuration. The loops 242, however, can include loop sections that lie in a plane in the expanded configuration. In the illustrated example, the first loop 242a includes first and second distal loop sections 266a, 268a and first apex 244a in a plane that is generally perpendicular to the axis A in the expanded configuration as distal planar loop section 280a. Further, the second loop 242b includes third and fourth distal loop sections 266b, 268b and second apex 244b in the same plane and are generally perpendicular to the axis A as distal planar loop section 280b. Loops 242a, 242a each include a pair distal bends 282a, 282b, respectively, proximate the respective distal planar loop sections 280a, 280b in the medial sections 270a, 272a, 270b, 272b turning the distal planar loop sections 280a, 280b toward the axis A. Additionally, loops 242a, 242b each include a pair of proximal bends 284a, 284b distal the ends 232a, 234a, 232b, 234b turning the medial sections 270a, 272a, 270b, 272b away from the axis A. In embodiments, electrodes from the set of the plurality of electrodes 250a, 250b are disposed on the distal planar loop sections 280a, 280b and can be disposed on the distal bends 282a, 282b. In one example, the electrodes 250 can be disposed on the straight portions of the loops 242, which can be constructed to be relatively more rigid sections of the loops 242, to provide for more robust tracking or locating of electrodes.

In the example, the electrode assembly 230 is configured to detect physiological signals from portions of the chamber walls in front of the catheter 200. The distal planar loop sections 280a, 280b of the non-planar loops 242a, 242b include first and second distal loop sections 266a, 268a and first apex 244a of the first loop 242a and the third and fourth distal loop sections 266b, 268b and second apex 244b of the second loop 242b, which include first and second electrodes 250a, 250b. As the distal planar loop sections 280a, 280b contact the chamber wall, the distal planar loop sections 280a, 280b remain generally planar as a force is applied along the axis A. Distal bends 282a, 282b in the medial sections 270, 272 of loops 242 can be configured to be relatively more flexible than the distal planar loop sections 280 and provide a flex point that deform or deflect under the force applied along the axis A. As the distal bends 282a, 282b in the medial section 270, 272 deform or deflect, the distal region 204 of the shaft 202, as well as the location sensor are moved closer to the electrodes 250 on the distal planar loop sections 270, 272, and a more accurate reading of location of the electrodes 250 can be detected. Furthermore, the geometry of the electrode assembly 220 having a section perpendicular to the axis A is more intuitive and better suited for control of the electrodes than, for example, a paddle-type electrode assembly in which the electrode assembly is in an axial plane.

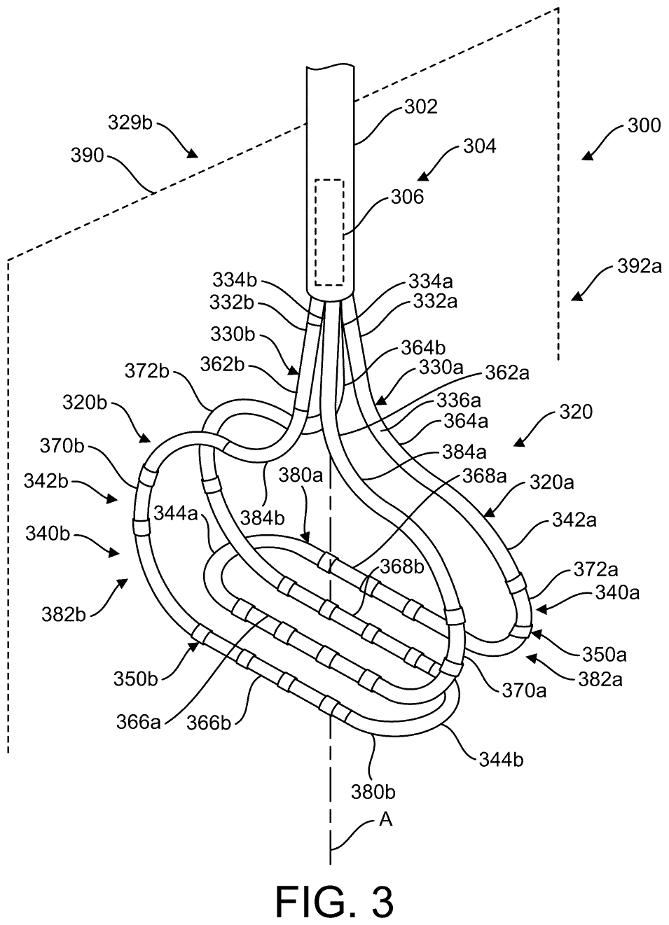

FIGS. 3-6 present alternative designs to catheter 200 and are illustrated in the expanded configuration. For example, the alternative designs can include electrode assemblies having loops configured with a distal planar section of four generally parallel support members, two sets of longitudinal planar sections of four generally parallel support members, or combination of distal planar sections and longitudinal planar sections. The designs can include sensing electrodes formed from ring electrodes and patch electrodes. Electrodes can also be configured as ablation electrodes or electrode assemblies can further include ablation electrode assemblies. For instance, ablation electrodes can be disposed on the planar sections of the electrode splines, which in many examples, can provide for ablation of the posterior wall. Further, the support members can be constructed to include varying degrees of flexibility or rigidity along the length of the spline or in selected areas of the spline. The loops can be flexed to provide for shapes to map with a matrix of electrodes and to move navigation sensors closer to the electrodes. Additionally, navigation sensors, including EAM electrodes, can be disposed on the loops and interspersed with the sensing electrodes. Concepts and features of catheter 200 can be applied to the alternative designs.

FIG. 3 is a partial perspective illustration of an embodiment of a catheter 300 configured to detect a plurality of physiological signals from within a patient's heart. The catheter 300 corresponds with the catheter 105 described with respect to FIG. 1 and can be used in the electrophysiology system 50. For example, catheter 300 can be configured as a mapping catheter in some embodiments and as a mapping and ablation catheter in some embodiments. The catheter 300 has an elongated shaft 302 having a proximal region and a shaft distal region 304 configured to be deployed proximate target tissue. The elongated shaft 302 defines a longitudinal axis A. In the illustrated embodiment, the catheter 302 includes a location sensor 306 (shown in phantom) coupled to the distal region 304. A plurality of sensing electrode assemblies 320 are coupled to the distal end region 304. For example, the plurality of sensing electrode assemblies 320 can include at least a first sensing electrode assembly 320a and a second sensing electrode assembly 320b.

The first sensing electrode assembly 320a includes a first spline 330a and a first plurality of electrodes 350a. The first spline 330a includes a first end 332a, an opposite, second end 334a, and a first intermediate portion 340a between the first end 332a and the second end 334a. The first spline 330a is formed as first non-planar loop 342a in the expanded configuration wherein the first and second ends 332a, 334a are coupled to the distal region 304 and first intermediate portion 340a extends from the distal region 304. The first plurality of electrodes 350a are disposed on the first intermediate portion 340a.

The second sensing electrode assembly 320b includes a second spline 330b and a second plurality of electrodes 350b. The second spline 330b includes a third end 332b, an opposite, fourth end 334b, and a second intermediate portion 340b between the third end 332b and the fourth end 334b. The second spline 330b is formed as second non-planar loop 342b in the expanded configuration wherein the third and fourth ends 332b, 334b are coupled to the distal region 304 and second intermediate portion 340b extends from the distal region 304. The second plurality of electrodes 350b are disposed on the second intermediate portion 340b. In the embodiments, the first intermediate portion 340a does not contact the second intermediate portion 340b in the expanded configuration.

The splines 330 are formed as non-planar loops 342 in the expanded configuration wherein the spline ends 332a, 334a, 332b, 334b are coupled to the distal region 304 and intermediate portions 340 extends from the distal region 304. Each of the loops 342a, 342b is terminated distally at an apex 344a 344b, respectively, on the associated intermediate portion 340a, 340b. The first loop 342a includes first and second proximal loop sections 362a, 364a distal the first and second spline ends 332a, 334a, respectively. The first loop 342a also includes first and second distal loop sections 366a, 368a proximal to the first apex 344a. The first loop 342a further includes first and second medial loop section 370a, 372a disposed between the first and second proximal loop sections 362a, 364a and the first and second distal loop sections 366a, 368a, respectively. The second loop 342b includes third and fourth proximal loop sections 362b, 364b distal the third and fourth spline ends 332b, 334b, respectively. The second loop 342b also include third and fourth distal loop sections 366b, 368b proximal to the second apex 344b. The second loop 342b further includes third and fourth medial loop section 370b, 372b disposed between the third and fourth proximal loop sections 362b, 364b and the third and fourth distal loop sections 366b, 368b, respectively.

In the embodiments, the first intermediate portion 340a does not contact the second intermediate portion 340b in the expanded configuration. For instance, the first and second distal loop sections 366a, 368a and first apex 344a are not coupled to the second loop 342b. And the second and third distal loop section 366b, 368b and second apex 344b are not coupled to the first loop 342a. In the embodiments, at least some of the first plurality of electrodes 350a are disposed on the first and second distal loop sections 366a, 368a, and at least some of the second plurality of electrode 350b are disposed on the third and fourth distal loop sections 366b, 368b.

The loops 342 are non-planar in the expanded configuration in that the proximal loop sections 362, 364 extend away from the axis A toward the medial sections 370, 372 in the expanded configuration; and the distal loop sections 366, 368 extend toward the axis A from the medial section 370, 372 in the expanded configuration. The loops 342, however, can include loop sections that lie in a plane in the expanded configuration. In the illustrated example, the first loop 342a includes first and second distal loop sections 366a, 368a and first apex 344a in a plane that is generally perpendicular to the axis A in the expanded configuration as distal planar loop section 380a. Further, the second loop 342b includes third and fourth distal loop sections 366b, 368b and second apex 344b in the same plane and are generally perpendicular to the axis A as distal planar loop section 380b. Loops 342a, 342a include distal bends 382a, 382b, respectively, proximate the respective distal planar loop sections 380a, 380b in the medial sections 370a, 372a, 370b, 372b. turning the distal planar loop sections 380a, 380b toward the axis A. Additionally, loops 342a, 342b each include a pair of proximal bends 384a, 384b distal the ends 332a, 334a, 332b, 334b turning the medial sections 370a, 372a, 370b, 372b away from the axis A. In embodiments, electrodes from the set of the plurality of electrodes 350a, 350b are disposed on the distal planar loop sections 380a, 380b and can be disposed on the bends 382a, 382b.

The electrode assemblies 320a, 320b are also characterized with respect to a reference plane 390 in which the axis A lies in the reference plane 390. Ends 332a 334a of the first spline 330a are disposed on a first side 392a of the reference plane 390, and ends 332b 334b of the second spline 330b are disposed on a second side 392b of the reference plane 390. The second side 392a is opposite the reference plane 390 for the first side 392a. In the illustrated embodiment, the distal bends 382a and proximal bends 384a on the first spline 330a are disposed on the first side 392a of the reference plane 390, and distal bends 382b and proximal bends 384b on the second spline 330b are disposed on the second side 392b of the reference plane 390. The first apex 344a of the first spline 330a is located on the second side 392b of the reference plane 390, such as underneath (distally along a reference axis parallel to axis A) one of the distal bends 382b of the second spline 330b. The second apex 344b of the second spline 330b is located on the first side 392a of the reference plane 390, such as underneath (distally along a reference axis parallel to axis A) one of the distal bends 382a of the first spline 330a. Accordingly, the distal planar loop sections 380a, 380b lie on both sides 392a, 392b of the reference plane 390.

In the illustrated example, the first and second splines 330a, 330b can be constructed as identical loops 342a, 342b in the expanded configuration that arranged with respect to the shaft 302 as forming opposing electrode assemblies 320a, 320b. The distal planar loop sections 390a, 390b can be arranged as intertwining, symmetric splines 330a, 330b. In another example, one of the first and second loops 342a, 342b can include further spaced-apart splines 330a, 330b than the other of the first and second loops 342a, 342b, i.e., one of the first and second loops 342a, 342b is wider than the other one in the expanded configuration. The loops 342a, 342b can be arranged to form opposing electrode assemblies 320a, 320b in which the narrow loop is within the wider loop and arranged in as asymmetric splines 330a, 330b.

In the illustrated example, the catheter 300 includes symmetric loops 342 having a single plane of electrodes 350, such as electrodes 350 disposed on relatively rigid portions of four support members 336 in a plane generally perpendicular to the axis A. The splines 330 in the expanded configuration can be flexed to move the tracking sensor 306 closer to the electrodes 350 in a conformed configuration, such as when pressed against a heart chamber wall.

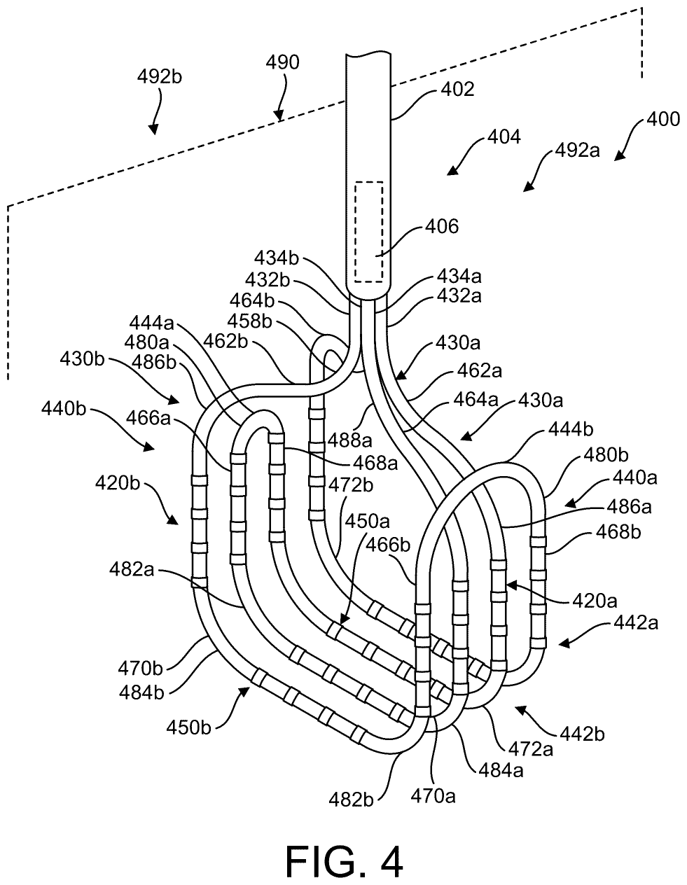

FIG. 4 is a partial perspective illustration of an embodiment of a catheter 400 configured to detect a plurality of physiological signals from within a patient's heart. The catheter 400 corresponds with the catheter 105 described with respect to FIG. 1 and can be used in the electrophysiology system 50. For example, catheter 400 can be configured as a mapping catheter in some embodiments and as a mapping and ablation catheter in some embodiments. The catheter 400 has an elongated shaft 402 having a proximal region and a shaft distal region 404 configured to be deployed proximate target tissue. The elongated shaft 402 defines a longitudinal axis A. In the illustrated embodiment, the catheter 402 includes a location sensor 406 (shown in phantom) coupled to the distal region 404. A plurality of sensing electrode assemblies 420 are coupled to the distal end region 404. For example, the plurality of sensing electrode assemblies 420 can include at least a first sensing electrode assembly 420a and a second sensing electrode assembly 420b.

The first sensing electrode assembly 420a includes a first spline 430a and a first plurality of electrodes 450a. The first spline 430a includes a first end 432a, an opposite, second end 434a, and a first intermediate portion 440a between the first end 432a and the second end 434a. The first spline 430a is formed as first non-planar loop 442a in the expanded configuration wherein the first and second ends 432a, 434a are coupled to the distal region 404 and first intermediate portion 440a extends from the distal region 404. The first plurality of electrodes 450a are disposed on the first intermediate portion 440a.

The second sensing electrode assembly 420b includes a second spline 430b and a second plurality of electrodes 450b. The second spline 430b includes a third end 432b, an opposite, fourth end 434b, and a second intermediate portion 440b between the third end 432b and the fourth end 434b. The second spline 430b is formed as second non-planar loop 442b in the expanded configuration wherein the third and fourth ends 432b, 434b are coupled to the distal region 404 and second intermediate portion 440b extends from the distal region 404. The second plurality of electrodes 450b are disposed on the second intermediate portion 440b. In the embodiments, the first intermediate portion 440a does not contact the second intermediate portion 440b in the expanded configuration.

The splines 430 are formed as non-planar loops 442 in the expanded configuration wherein the spline ends 432a, 434a, 432b, 434b are coupled to the distal region 404 and intermediate portions 440 extends from the distal region 404. Each of the loops 442a, 442b is terminated distally at an apex 444a 444b, respectively, on the associated intermediate portion 440a, 440b. The first loop 442a includes first and second proximal loop sections 462a, 464a distal the first and second spline ends 432a, 434a, respectively. The first loop 442a also includes first and second distal loop sections 466a, 468a proximal to the first apex 444a. The first loop 442a further includes first and second medial loop sections 470a, 472a disposed between the first and second proximal loop sections 462a, 464a and the first and second distal loop sections 466a, 468a, respectively. The second loop 442b includes third and fourth proximal loop sections 462b, 464b distal the third and fourth spline ends 432b, 434b, respectively. The second loop 442b also include third and fourth distal loop sections 466b, 468b proximal to the second apex 444b. The second loop 442b further includes third and fourth medial loop section 470b, 472b disposed between the third and fourth proximal loop sections 462b, 464b and the third and fourth distal loop sections 466b, 468b, respectively.

In the embodiments, the first intermediate portion 440a does not contact the second intermediate portion 440b in the expanded configuration. For instance, the first and second distal loop sections 466a, 468a and first apex 444a are not coupled to the second loop 442b. And the second and third distal loop section 466b, 468b and second apex 444b are not coupled to the first loop 442a.

The loops 442 are non-planar in the expanded configuration in that the proximal loop sections 462, 464 extend away from the axis A toward the medial sections 470, 472 in the expanded configuration and are configured in a generally opposing rectangular-hook arrangements. The loops 442, however, can include loop sections that lie in multiple planes in the expanded configuration. In the illustrated example, the first loop 442a includes first and second distal loop sections 466a, 468a and first apex 444a in a first plane that is generally parallel to the axis A in the expanded configuration as distal planar loop section 480a. The first loop 442a includes first and second medial loop sections 470a, 472a in a second plane that is generally perpendicular to the axis A in the expanded configuration. The first loop 442a also includes first and second proximal loop sections 462a, 464a in a third plane that is generally parallel to and spaced apart from the first plane across the axis A. Further, the second loop 442b includes third and fourth distal loop sections 466b, 468b and second apex 444b in the third plane with first and second proximal loop sections 462a, 464a. The second loop 442b includes third and fourth medial loop sections 470b, 472b in the second plane with first and second medial loop section 470a, 470b. The second loop 442b also includes third and fourth proximal loop sections 462b, 464b in the third plane with first and second distal loop section 466a, 468b.

Loop 442a, includes first distal bends 482a proximate the first and second distal loop sections 466a, 468a; first distal-medial bends 484a proximate to the first and second medial loop sections 470a, 472a; first proximal-medial bends 486a in the first and second proximal loop section 462a, 464a; and first proximal bends 488a in the first and second proximal loop sections 462a, 464a and proximal to the first proximal-medial bends 486a. Loop 442b includes second distal bends 482b proximate the third and fourth distal loop sections 466b, 468b; second distal-medial bends 484b proximate to the third and fourth medial loop sections 470b, 472b; second proximal-medial bends 486b in the third and fourth proximal loop sections 462b, 464b; and second proximal bends 488b in the third and fourth proximal loop section 462b, 464b and proximal to the second proximal-medial bends 486b.

In embodiments, electrodes from the set of the plurality of electrodes 450a, 450b are disposed on the proximal planar loop sections 462, 464, distal loop sections 466, 468, and medial loop sections 470, 472 forming three planes of electrodes. In particular, the electrodes 450 on the first and second distal loop sections 466a, 468a and first apex 444a of the first loop 442a and third and fourth proximal loop sections 462b, 464b of the second loop 442b lie in the first plane. Electrodes 450 on the first and second medial loop sections 470a, 470b of the first loop 442a and third and fourth medial loop section 470b, 472b of the second loop 442b lie in the second plane. Electrodes 450 on the first first and second proximal portions 462a, 464a of the first loop 442a and third and fourth distal loop sections 466b, 468b and second apex 444b of the second loop 442b lie in the third plane.

The electrode assemblies 420a, 420b are also characterized with respect to a reference plane 490 in which the axis A lies in the reference plane 490. Ends 432a 434a of the first spline 430a are disposed on a first side 492a of the reference plane 490, and ends 432b 434b of the second spline 430b are disposed on a second side 492b of the reference plane 490. The second side 492a is opposite the reference plane 490 for the first side 492. The first apex 444a of the first spline 430a is located on the second side 492b of the reference plane 490. The second apex 444b of the second spline 430b is located on the first side 492a of the reference plane 490.

In the illustrated example, the first splines 430a are formed in a wider loop 442a than the second loop 442b, and the loops are asymmetrical in the expanded configuration. The loops 442a, 442b are arranged to form opposing electrode assemblies 420a, 420b in which the narrow second loop 442b is within the wider first loop 442b. Alternatively, the first and second splines 430a, 430b can be constructed as identical loops 442a, 442b in the expanded configuration that arranged with respect to the shaft 402 as forming opposing electrode assemblies 420a, 420b. The distal planar loop sections 480a, 480b can be arranged as intertwining, symmetric splines 430a, 430b.

In the illustrated example, the catheter 400 includes asymmetric loops 442 having three planes of electrodes 450, such as electrodes 450 disposed on relatively rigid portions of four support members 436 in a plane generally perpendicular to the axis A and two planes generally parallel to the axis A. The splines 430 in the expanded configuration can be flexed to move the tracking sensor 406 closer to the electrodes 450 in a conformed configuration, such as when pressed against a heart chamber wall.

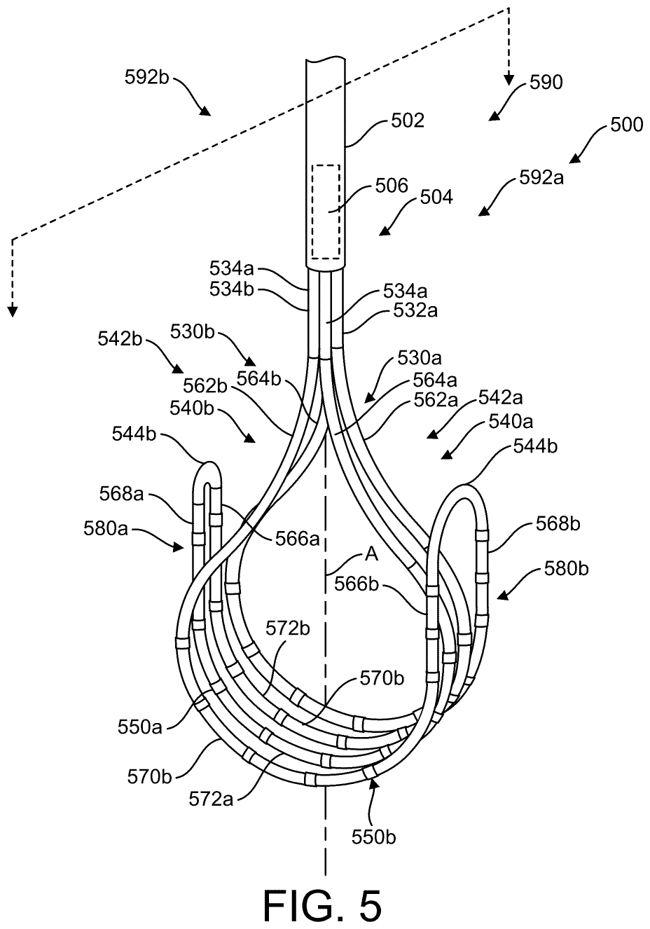

FIG. 5 is a partial perspective illustration of an embodiment of a catheter 500 configured to detect a plurality of physiological signals from within a patient's heart. The catheter 500 corresponds with the catheter 105 described with respect to FIG. 1 and can be used in the electrophysiology system 50. For example, catheter 500 can be configured as a mapping catheter in some embodiments and as a mapping and ablation catheter in some embodiments. The catheter 500 has an elongated shaft 502 having a proximal region and a shaft distal region 504 configured to be deployed proximate target tissue. The elongated shaft 502 defines a longitudinal axis A. In the illustrated embodiment, the catheter 502 includes a location sensor 506 (shown in phantom) coupled to the distal region 504. A plurality of sensing electrode assemblies 520 are coupled to the distal end region 504. For example, the plurality of sensing electrode assemblies 520 can include at least a first sensing electrode assembly 520a and a second sensing electrode assembly 520b.

The first sensing electrode assembly 520a includes a first spline 530a and a first plurality of electrodes 550a. The first spline 530a includes a first end 532a, an opposite, second end 534a, and a first intermediate portion 540a between the first end 532a and the second end 534a. The first spline 530a is formed as first non-planar loop 542a in the expanded configuration wherein the first and second ends 532a, 534a are coupled to the distal region 504 and first intermediate portion 540a extends from the distal region 504. The first plurality of electrodes 550a are disposed on the first intermediate portion 540a.

The second sensing electrode assembly 520b includes a second spline 530b and a second plurality of electrodes 550b. The second spline 530b includes a third end 532b, an opposite, fourth end 534b, and a second intermediate portion 540b between the third end 532b and the fourth end 534b. The second spline 530b is formed as second non-planar loop 542b in the expanded configuration wherein the third and fourth ends 532b, 534b are coupled to the distal region 504 and second intermediate portion 540b extends from the distal region 504. The second plurality of electrodes 550b are disposed on the second intermediate portion 540b. In the embodiments, the first intermediate portion 540a does not contact the second intermediate portion 540b in the expanded configuration.

The splines 530 are formed as non-planar loops 542 in the expanded configuration wherein the spline ends 532a, 534a, 532b, 534b are coupled to the distal region 504 and intermediate portions 540 extends from the distal region 504. Each of the loops 542a, 542b is terminated distally at an apex 544a 544b, respectively, on the associated intermediate portion 540a, 540b. The first loop 542a includes first and second proximal loop sections 562a, 564a distal the first and second spline ends 532a, 534a, respectively. The first loop 542a also includes first and second distal loop sections 566a, 568a proximal to the first apex 544a. The first loop 542a further includes first and second medial loop sections 570a, 572a disposed between the first and second proximal loop sections 562a, 564a and the first and second distal loop sections 566a, 568a, respectively. The second loop 542b includes third and fourth proximal loop sections 562b, 564b distal the third and fourth spline ends 532b, 534b, respectively. The second loop 542b also include third and fourth distal loop sections 566b, 568b proximal to the second apex 544b. The second loop 542b further includes third and fourth medial loop section 570b, 572b disposed between the third and fourth proximal loop sections 562b, 564b and the third and fourth distal loop sections 566b, 568b, respectively.

In the embodiments, the first intermediate portion 540a does not contact the second intermediate portion 540b in the expanded configuration. For instance, the first and second distal loop sections 566a, 568a and first apex 544a are not coupled to the second loop 542b. And the second and third distal loop section 566b, 568b and second apex 544b are not coupled to the first loop 542a. In the embodiments, at least some of the first plurality of electrodes 550a are disposed on the first and second medial sections 570a, 572a and first and second distal loop sections 566a, 468a, and at least some of the second plurality of electrode 550b are disposed on the third and fourth medial loop sections 570b, 572b and third and fourth distal loop sections 566b, 568b.

The loops 542 are non-planar in the expanded configuration in that the proximal loop sections 562, 564 extend away from the axis A toward the medial sections 570, 572 in the expanded configuration and are configured in a generally opposing curvilinear-hook arrangements. The loops 542, however, can include loop sections that lie in multiple planes in the expanded configuration. In the illustrated example, the first loop 542a includes first and second distal loop sections 566a, 568a and first apex 544a in a first plane that is generally parallel to the axis A in the expanded configuration as distal planar loop section 580a. The first loop 542a includes first and second medial loop sections 570a, 572a in a curved or cylindrical form in the expanded configuration. The first loop 542a also includes first and second proximal loop sections 562a, 564a in a curved or cylindrical form and spaced apart from the first plane across the axis A. Further, the second loop 542b includes third and fourth distal loop sections 566b, 568b and second apex 544b in a second plane that is generally parallel to the axis A in the expanded configuration as distal planar loop section 580b and spaced apart across the axis A from the first plane 580a. The second loop 542b includes third and fourth medial loop sections 570b, 572b in the curved or cylindrical form of first and second medial loop section 570a, 570b. The second loop 542b also includes third and fourth proximal loop sections 562b, 564b in the curved or cylindrical with first and second distal loop section 566a, 568b.

First loop 542a, includes first distal bends 582a in the first and second medial loop sections 570a, 572a and first proximal bends 484a in the first and second proximal loop sections 562a, 564a. Second loop 542b includes second distal bends 582b in the third and fourth medial loop sections 570b, 572b and second proximal bends 548a in the third and fourth proximal loop section 462b, 464b.

In embodiments, electrodes from the set of the plurality of electrodes 550a, 550b are disposed on the proximal planar loop sections 562, 564, distal loop sections 566, 568, and medial loop sections 570, 572 forming two planes of electrodes amongst curved or cylindrical portions. In particular, the electrodes 550 on the first and second distal loop sections 566a, 568a and first apex 544a of the first loop 542a lie in the first plane. Electrodes 550 on the third and fourth distal loop sections 566b, 568b and second apex 544b of the second loop lie in the second plane. Electrodes 450 on the first and second proximal loop section 562a, 564a and first and second medial loop sections 470a, 470b of the first loop 542a and third and fourth proximal sections 562b, 564b and third and fourth medial loop sections 470b, 472b of second loop 542b lie in a curved or cylindrical portion.

The electrode assemblies 520a, 520b are also characterized with respect to a reference plane 590 in which the axis A lies in the reference plane 590. Ends 532a 534a of the first spline 530a are disposed on a first side 592a of the reference plane 590, and ends 532b 534b of the second spline 530b are disposed on a second side 592b of the reference plane 590. The second side 592a is opposite the reference plane 590 for the first side 592. The first apex 544a of the first spline 530a is located on the second side 592b of the reference plane 590. The second apex 544b of the second spline 530b is located on the first side 592a of the reference plane 590.

In the illustrated example, the first splines 530a are formed in a wider loop 542a than the second loop 542b, and the loops 542a, 542b are asymmetrical in the expanded configuration. The loops 542a, 542b are arranged to form opposing electrode assemblies 520a, 520b in which the narrow second loop 542b is within the wider first loop 542b. Alternatively, the first and second splines 530a, 530b can be constructed as identical loops 542a, 542b in the expanded configuration that arranged with respect to the shaft 502 as forming opposing electrode assemblies 520a, 520b.

In the illustrated example, the catheter 500 includes asymmetric loops 542 having two parallel planes of electrodes 550, such as electrodes 350 disposed on relatively rigid portions of four support members 536 in planes generally parallel to the axis A with rounded distal portions of the electrode assemblies 520. The splines 530 in the expanded configuration can be flexed to move the tracking sensor 506 closer to the electrodes 550 in a conformed configuration, such as when pressed against a heart chamber wall.

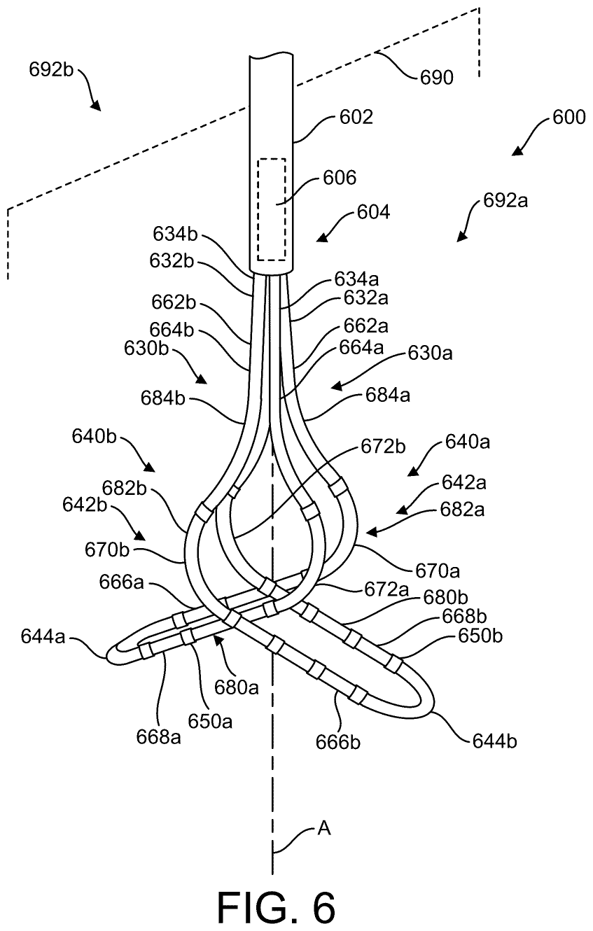

FIG. 6 is a partial perspective illustration of an embodiment of a catheter 600 configured to detect a plurality of physiological signals from within a patient's heart. The catheter 600 corresponds with the catheter 105 described with respect to FIG. 1 and can be used in the electrophysiology system 50. For example, catheter 600 can be configured as a mapping catheter in some embodiments and as a mapping and ablation catheter in some embodiments. The catheter 600 has an elongated shaft 602 having a proximal region and a shaft distal region 604 configured to be deployed proximate target tissue. The elongated shaft 602 defines a longitudinal axis A. In the illustrated embodiment, the catheter 602 includes a location sensor 606 (shown in phantom) coupled to the distal region 604. A plurality of sensing electrode assemblies 620 are coupled to the distal end region 604. For example, the plurality of sensing electrode assemblies 620 can include at least a first sensing electrode assembly 620a and a second sensing electrode assembly 620b.

The first sensing electrode assembly 620a includes a first spline 630a and a first plurality of electrodes 650a. The first spline 630a includes a first end 632a, an opposite, second end 634a, and a first intermediate portion 640a between the first end 632a and the second end 634a. The first spline 630a is formed as first non-planar loop 642a in the expanded configuration wherein the first and second ends 632a, 634a are coupled to the distal region 604 and first intermediate portion 640a extends from the distal region 604. The first plurality of electrodes 650a are disposed on the first intermediate portion 640a.

The second sensing electrode assembly 620b includes a second spline 630b and a second plurality of electrodes 650b. The second spline 630b includes a third end 632b, an opposite, fourth end 634b, and a second intermediate portion 640b between the third end 632b and the fourth end 634b. The second spline 630b is formed as second non-planar loop 642b in the expanded configuration wherein the third and fourth ends 632b, 634b are coupled to the distal region 604 and second intermediate portion 640b extends from the distal region 604. The second plurality of electrodes 650b are disposed on the second intermediate portion 640b. In the embodiments, the first intermediate portion 640a does not contact the second intermediate portion 640b in the expanded configuration.

The splines 630 are formed as non-planar loops 642 in the expanded configuration wherein the spline ends 632a, 634a, 632b, 634b are coupled to the distal region 604 and intermediate portions 640 extends from the distal region 604. Each of the loops 642a, 642b is terminated distally at an apex 644a 644b, respectively, on the associated intermediate portion 640a, 640b. The first loop 642a includes first and second proximal loop sections 662a, 664a distal the first and second spline ends 632a, 634a, respectively. The first loop 642a also includes first and second distal loop sections 666a, 668a proximal to the first apex 644a. The first loop 642a further includes first and second medial loop section 670a, 672a disposed between the first and second proximal loop sections 662a, 664a and the first and second distal loop sections 666a, 668a, respectively. The second loop 642b includes third and fourth proximal loop sections 662b, 664b distal the third and fourth spline ends 632b, 634b, respectively. The second loop 642b also include third and fourth distal loop sections 666b, 668b proximal to the second apex 644b. The second loop 642b further includes third and fourth medial loop section 670b, 672b disposed between the third and fourth proximal loop sections 662b, 664b and the third and fourth distal loop sections 666b, 668b, respectively.

In the embodiments, the first intermediate portion 640a does not contact the second intermediate portion 640b in the expanded configuration. For instance, the first and second distal loop sections 666a, 668a and first apex 644a are not coupled to the second loop 642b. And the second and third distal loop section 666b, 668b and second apex 644b are not coupled to the first loop 642a. In the embodiments, at least some of the first plurality of electrodes 650a are disposed on the first and second distal loop sections 666a, 668a, and at least some of the second plurality of electrode 650b are disposed on the third and fourth distal loop sections 666b, 668b.

The loops 642 are non-planar in the expanded configuration in that the proximal loop sections 662, 664 extend away from the axis A toward the medial sections 670, 672 in the expanded configuration; and the distal loop sections 666, 668 extend toward the axis A from the medial section 670, 672 in the expanded configuration. The loops 642, however, can include loop sections that lie in planes in the expanded configuration. In the illustrated example, the first loop 642a includes first and second distal loop sections 666a, 668a and first apex 644a in a first plane that is generally non-perpendicular and non-parallel to a plane of the axis A in the expanded configuration as distal planar loop section 680a. Further, the second loop 642b includes third and fourth distal loop sections 666b, 668b and second apex 644b lie in a second plane that is also generally non-perpendicular and non-parallel to the plane of the axis A as distal planar loop section 380b. Distal planar loop section 680a is generally opposite distal planar loop section 680b. Loops 642a, 642a include distal bends 682a, 682b, respectively, proximate the respective distal planar loop sections 680a, 680b in the medial sections 670a, 672a, 670b, 672b. turning the distal planar loop sections 680a, 680b toward the axis A. Additionally, loops 642a, 642b each include a pair of proximal bends 684a, 684b distal the ends 632a, 634a, 632b, 634b turning the medial sections 670a, 672a, 670b, 672b away from the axis A. In embodiments, electrodes from the set of the plurality of electrodes 650a, 650b are disposed on the distal planar loop sections 680a, 680b and can be disposed on the bends 682a, 682b.

The electrode assemblies 620a, 620b are also characterized with respect to a reference plane 690 in which the axis A lies in the reference plane 690. Ends 632a 634a of the first spline 630a are disposed on a first side 692a of the reference plane 690, and ends 632b 634b of the second spline 630b are disposed on a second side 692b of the reference plane 690. The second side 692a is opposite the reference plane 690 for the first side 692. In the illustrated embodiment, the distal bends 682a and proximal bends 684a on the first spline 630a are disposed on the first side 692a of the reference plane 690, and distal bends 682b and proximal bends 684b on the second spline 630b are disposed on the second side 692b of the reference plane 690. The first apex 644a of the first spline 630a is located on the second side 692b of the reference plane 690, such as underneath (distally along a reference axis parallel to axis A) one of the distal bends 682b of the second spline 630b. The second apex 644b of the second spline 330b is located on the first side 692a of the reference plane 690, such as underneath (distally along a reference axis parallel to axis A) one of the distal bends 682a of the first spline 630a. Accordingly, the distal planar loop sections 680a, 680b lie on both sides 692a, 692b of the reference plane 690.

In the illustrated example, the first and second splines 630a, 630b can be constructed as identical loops 642a, 642b in the expanded configuration that arranged with respect to the shaft 602 as forming opposing electrode assemblies 620a, 620b. The distal planar loop sections 690a, 690b can be arranged as intertwining, symmetric splines 630a, 630b. In another example, one of the first and second loops 642a, 642b can include further spaced-apart splines 630a, 630b than the other of the first and second loops 642a, 642b, i.e., one of the first and second loops 642a, 642b is wider than the other one in the expanded configuration. The loops 642a, 642b can be arranged to form opposing electrode assemblies 620a, 620b in which the narrow loop is within the wider loop and arranged in as asymmetric splines 630a, 630b.

In the illustrated example, the catheter 600 includes symmetric loops 642 having two planes of electrodes 650, such as electrodes 650 disposed on relatively rigid portions of two support members each that can be flexed or deflected into a plane generally perpendicular to the axis A with four support members 636. The splines 630 in the expanded configuration can be configured to be highly flexible and conform to posterior surface of the heart chamber and to move the tracking sensor 606 closer to the electrodes 650 in a conformed configuration.

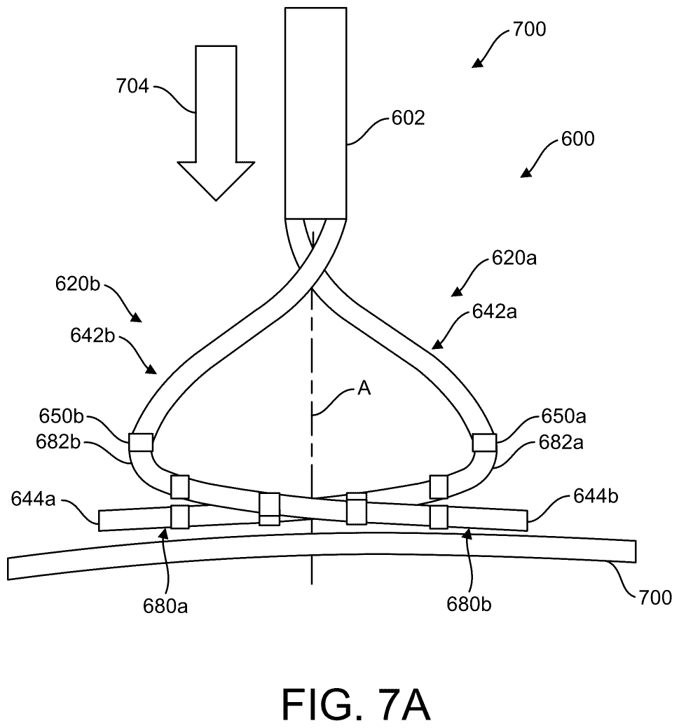

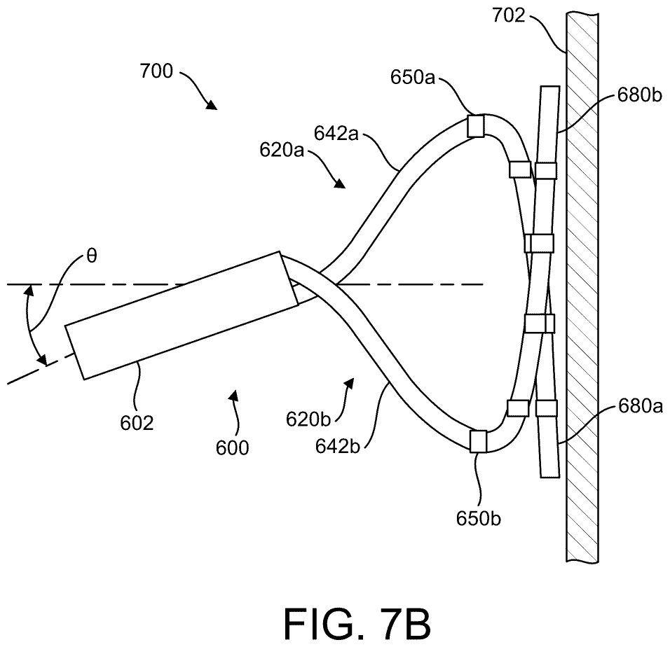

FIGS. 7A and 7B illustrate an example catheter, such as catheter 600, while in a mapping operation in a heart chamber 700. Aspects of the operation can extend to other catheters, such as catheters 200-500, as well. The catheter 600 gains access to the chamber 700 through the vasculature, such as in a collapsed configuration within a delivery sheath. The delivery sheath can be retracted, or the catheter 600 can be extended from the sheath, and the catheter 600 can be deployed in an expanded configuration such as that illustrated in FIG. 6. In many respects, anatomical mapping and high-density mapping operations can be performed similarly to basket catheters. For instance, in the expanded configuration, the catheter 600 can map with all electrodes 650a, 650b. The geometry of the electrode assemblies 620a, 620b can be created through such techniques as telescoping, rotating, sheath manipulation, and deflecting the catheter from the expanded configuration. FIG. 7A illustrates the catheter 600 in a conformed configuration from the expanded configuration. The distal end portions 680a, 680b of the loops 642a, 642b are pressed against a posterior wall 702 of the chamber 700. The catheter shaft 602 is urged in the direction of arrow 704 along axis A toward the posterior wall 702, and the resulting force causes the catheter 600 to deform or deflect at least at distal bends 682a, 682b to move the distal end portions 680a, 680b towards a flat shape of the posterior wall 702. In this conformed configuration, the catheter 600 presents a high-density matrix of electrodes 650a, 650b from loops 642a, 642b against the posterior wall for high-density mapping. FIG. 7B illustrates the electrode assemblies 620a, 620b can also be angled and flexed from axis of the shaft 602 during the mapping operation. For instance, deflection and steering controls in the shaft 602 can tilt off-axis the catheter 600 in the expanded configuration or in the conformed configuration, as shown, by an angle θ to gain proper access of the posterior wall 702 and maintain the shape of the distal end portions 680a, 680b in a high-density electrode matrix.

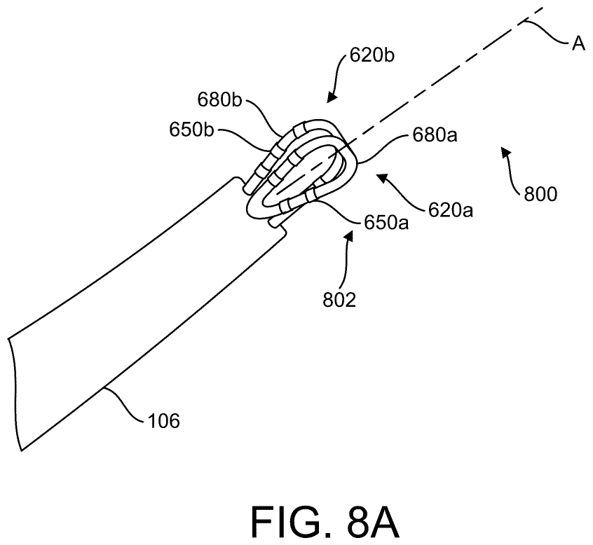

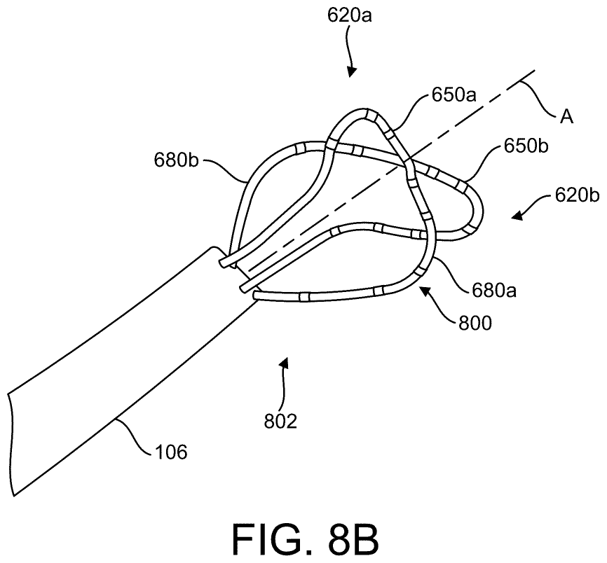

FIGS. 8A and 8B illustrate an example catheter, such as catheter 600, while in operation in a pulmonary vein 800. Aspects of the operation can extend to other catheters, such as catheters 200-500, as well. The catheter 600 gains access to the pulmonary vein 800 through the vasculature, such as in a collapsed configuration within a delivery sheath 106. As illustrated in FIG. 8A, the catheter 600 is navigated to the pulmonary vessel ostium 802 in the collapsed configuration within the sheath 106 or partially collapsed configuration with distal end portions 680a, 680b of the electrode assemblies 620a, 620b partially extending from the sheath 106 as shown. As illustrated in FIG. 8B, the delivery sheath 106 is retracted partially retracted, or the catheter 600 is extended or partially extended from the sheath 106, and the catheter 600 can be deployed in the vessel ostium 802 such that the distal end portions 680a, 680b expand such that electrodes 650a, 650b contact tissue of the vessel 800. The electrode assemblies 620a, 620b can be rotated about axis A to map the vessels 800.

The catheters 200, 300, 400, 500, 600 share several attributes. Among these attributes are the catheters 200-600 can deform or deflect from the expanded configuration to form a flat surface with electrodes close to the tracking sensor to provide robust mapping in the conformed configuration. The catheters 200-600 provide for volumetric forms having distal sections that provide a more intuitive orientation for handling than paddle catheters. The illustrated shapes are simple and structurally consistent and fold into the collapsed configuration with relative ease.

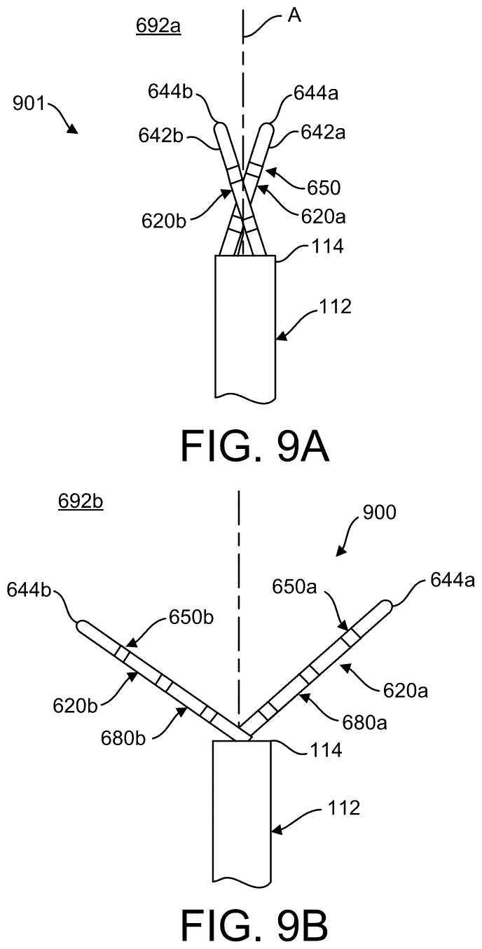

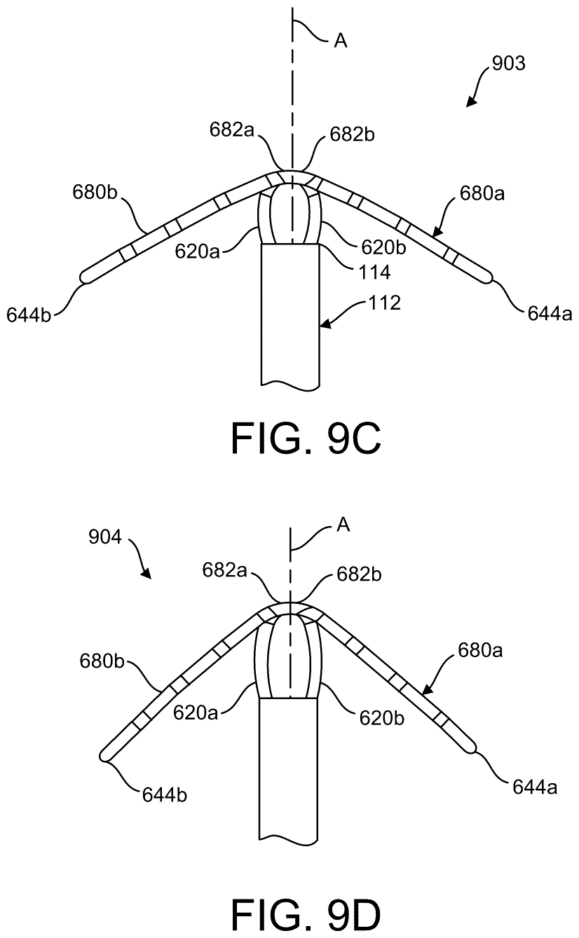

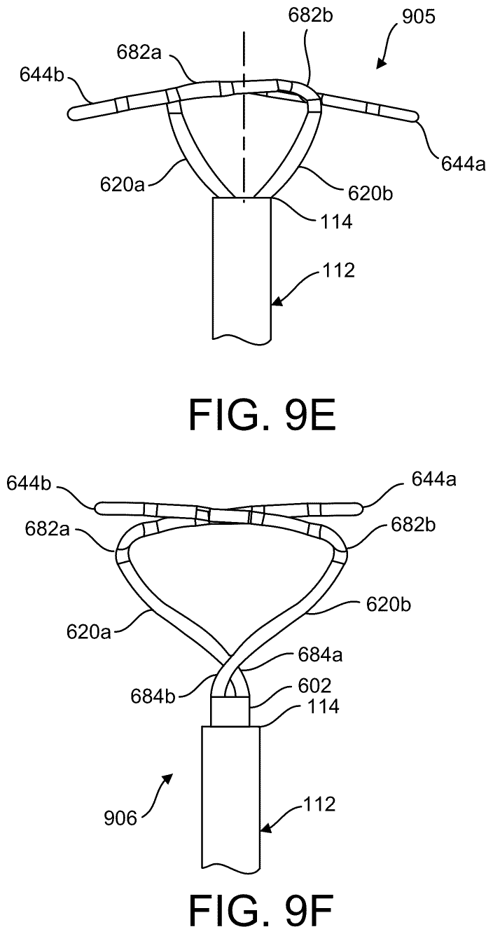

FIGS. 9A-9F illustrate various stages of the sensing electrode assemblies 620 catheter 600 transitioning from the collapsed state to an expanded state. In embodiments, the sensing electrode assemblies 620 are arranged in the collapsed configuration to fit within a catheter sheath, such as sheath 112 having a sheath distal tip 114. For example, the catheter 600 can assume the collapsed configuration to travel through a patient's vasculature, or within an opening in tissue such as a puncture in the atrial septum, to a therapy site. The sensing electrode assemblies 620 are configured to self-expand from the collapsed configuration when constrained within a delivery sheath to the pre-defined expanded configuration.

FIG. 9A illustrates the configuration of the sensing electrode assemblies 620a, 620b in a first stage 901 of the catheter 600 extended from the sheath 112. In the illustrated embodiment, the first and second apexes 644a, 644b of the first and second loops 642a, 642b, respectively are first to emerge from the sheath 112 as the catheter 600 is extended from the sheath distal tip 114. The sensing electrode assemblies 620a, 620 already begin to cross the reference plane include axis A from a first side 692a to a second side 692b or vice versa.

FIG. 9B illustrates the configuration of the sensing electrode assemblies 620a, 620b in a second stage 902 of the catheter 600 extended from the sheath 112. In the illustrated embodiment, the first and second apexes 644a, 644b of the first and second loops 642a, 642b, respectively, spread apart laterally as the sensing electrode assemblies 620a, 620b continue to emerge from the sheath 112. As distal end portions 680a, 680b are extended from the sheath 112, the electrode assemblies 620a, 620b are urged from opposite sides of the inner wall of the sheath 112 and across the axis A. The distal end portions 680a, 680 in the second stage 902 extend distally from the sheath distal tip 114. In the second stage 902, the distal bends 682a, 682b have yet to emerge from under the sheath 112.

FIGS. 9C, 9D, and 9E illustrate the configuration of the sensing electrode assemblies 620a, 620b in a third stage 903, fourth stage 904, and fifth stage 905 of the catheter 600 extended from the sheath 112. In the illustrated embodiment, the distal bends 682a, 682b have emerged from the sheath 112. The distal end portions 680a, 680b and apexes 644a, 644b now extend and point proximally from the sheath distal tip 114. As the sensing electrode assemblies 620a, 620b move from the third stage 903 to the fourth stage 904, the distal end portions 680a, 680b move toward the axis A and are angled further proximally. As the sensing electrode assemblies 620a, 620b move from the fourth stage 904 to the fifth stage 905, however, the distal end portions 680a, 680b and apexes 644a, 644b begin to extend distally from their positions in the fourth stage 904 as the distal bends 682a, 682b are extended longitudinally from the sheath 112. Also, the distal bends 682a, 682b are spread apart laterally as the sensing electrode assemblies 620a, 620b move from the fourth stage 904 to the fifth stage 905. The proximal bends 684a, 684b have yet to emerge from under the sheath in the fifth stage 905.

FIG. 9F illustrates the sensing electrode assemblies 620a, 620b in a sixth stage 906, or in the expanded configuration. The proximal bends 684a, 684b and spline ends 632a, 632b, 634a, 634b have emerged from the sheath 112, and no portion of the electrode assemblies 620a, 620b is within the sheath 112 in the illustrated embodiment. In one embodiment, the catheter 600 reverses the stages, or proceeds from stage 906 to 901, as the catheter 600 transitions from the expanded configuration to the collapsed configuration.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A catheter to detect a plurality of physiological signal from within a patient's heart, the catheter comprising:
an elongated shaft having a proximal region and a distal region, the elongated shaft defining a longitudinal axis;
a location sensor coupled to the distal region and configured to generate a plurality of location signals representative of a location of the catheter within the patient's heart; and
a plurality of sensing electrode arrays coupled to the distal region, the plurality of sensing electrode arrays having a collapsed configuration and an expanded configuration, the plurality of sensing electrode arrays including a first sensing electrode array and a second sensing electrode array;
the first sensing electrode array, comprising:
a first spline having a first end, an opposite, second end, and a first intermediate portion between the first end and the second end, the first spline formed as first non-planar loop in the expanded configuration wherein the first and second ends are coupled to the distal region of the shaft and first intermediate portion extends from the distal region; and
a first plurality of electrodes disposed on the first intermediate portion;
the second sensing electrode array, comprising:
a second spline having a third end, an opposite, fourth end, and a second intermediate portion between the third end and the fourth end, the second spline formed as second non-planar loop in the expanded configuration wherein the third and fourth ends are coupled to the distal region of the shaft and second intermediate portion extends from the distal region; and
a second plurality of electrodes disposed on the second intermediate portion;
wherein the first intermediate portion does not contact the second intermediate portion in the expanded configuration.

2. The catheter of claim 1, wherein the plurality of sensing electrode arrays includes two sensing electrode arrays.

3. The catheter of claim 1, wherein the plurality of sensing electrode arrays are deformable from the expanded configuration.

4. The catheter of claim 3, wherein the first non-planar loop includes first bends in the first spline and the second non-planar loop includes second bends in the second spline, the first and second bends configured to flex when deformed from the expanded configuration.

5. The catheter of claim 4, wherein the first bends include a first distal bends and first proximal bends, and the second bends include second distal bends and second proximal bends.

6. The catheter of claim 1, wherein the first non-planar loop includes a first distal planar loop section in a first plane and the second non-planar loop includes a second distal planar loop section in a second plane in the expanded configuration.

7. The catheter of claim 6, wherein the first plane lies in the second plane in the expanded configuration, the first and second planes perpendicular to the longitudinal axis.

8. The catheter of claim 6, wherein the first planar loop section and second planar loop section are deflectable from an expanded configuration such that the first plane lies in the second plane, the first and second planes perpendicular to the longitudinal axis.

9. The catheter of claim 6, wherein the first plane is parallel to the second plane in the expanded configuration, the first and second planes parallel to a reference plane including the longitudinal axis.

10. The catheter of claim 9, wherein the first non-planar loop includes a first medial planar loop section in a third plane and the second non-planar loop includes a second medial loop section in the third plane, the third plane perpendicular to the longitudinal axis in the expanded configuration.

11. The catheter of claim 1, wherein the first non-planar loop extends from a first side to a second side of a reference plane including the axis, and the second non-planar loop extends from a second side to a first side of the reference plane in the expanded configuration.

12. The catheter of claim 1, wherein the location sensor is disposed in the shaft.

13. The catheter of claim 1, wherein the plurality of sensing arrays are configured to tilt off axis from the shaft.

14. An electrophysiological system, comprising:
a catheter to detect a plurality of physiological signal from within a patient's heart, the catheter comprising:
an elongated shaft having a proximal region and a distal region, the elongated shaft defining a longitudinal axis;
a location sensor coupled to the distal region and configured to generate a plurality of location signals representative of a location of the catheter within the patient's heart; and
a plurality of sensing electrode arrays coupled to the distal region, the plurality of sensing electrode arrays having a collapsed configuration and an expanded configuration, the plurality of sensing electrode arrays including a first sensing electrode array and a second sensing electrode array;
the first sensing electrode array, comprising:
a first spline having a first end, an opposite, second end, and a first intermediate portion between the first end and the second end, the first spline formed as first non-planar loop in the expanded configuration wherein the first and second ends are coupled to the distal region of the shaft and first intermediate portion extends from the distal region; and
a first plurality of electrodes disposed on the first intermediate portion;
the second sensing electrode array, comprising:
a second spline having a third end, an opposite, fourth end, and a second intermediate portion between the third end and the fourth end, the second spline formed as second non-planar loop in the expanded configuration wherein the third and fourth ends are coupled to the distal region of the shaft and second intermediate portion extends from the distal region; and
a second plurality of electrodes disposed on the second intermediate portion;
wherein the first intermediate portion does not contact the second intermediate portion in the expanded configuration; and
an electroanatomical mapping system coupled to the catheter and configured to receive signals representative of the detected physiological signals from the plurality of sensing electrode arrays and map the patient's heart.

15. The electrophysiological system of claim 14, and further comprising a console coupled to the catheter, the console configured to provide ablation energy to the catheter.

16. A catheter to detect a plurality of physiological signal from within a patient's heart, the catheter comprising:
an elongated shaft having a proximal region and a distal region, the elongated shaft defining a longitudinal axis;
a location sensor coupled to the distal region and configured to generate a plurality of location signals representative of a location of the catheter within the patient's heart; and
a pair of sensing electrode arrays coupled to the distal region, the pair of sensing electrode arrays having a collapsed configuration and an expanded configuration, the pair of sensing electrode arrays including a first sensing electrode array and a second sensing electrode array;
the first sensing electrode array, comprising:
a first spline having a first end, an opposite, second end, and a first intermediate portion between the first end and the second end, the first spline formed as first non-planar loop in the expanded configuration wherein the first and second ends are coupled to the distal region of the shaft and first intermediate portion extends from the distal region; and
a first plurality of electrodes disposed on the first intermediate portion;
the second sensing electrode array, comprising:
a second spline having a third end, an opposite, fourth end, and a second intermediate portion between the third end and the fourth end, the second spline formed as second non-planar loop in the expanded configuration wherein the third and fourth ends are coupled to the distal region of the shaft and second intermediate portion extends from the distal region, the second spline opposite the first spline; and
a second plurality of electrodes disposed on the second intermediate portion;
wherein the first intermediate portion does not contact the second intermediate portion in the expanded configuration.

17. The catheter of claim 16, wherein the first non-planar loop includes a first distal planar loop section in a first plane and the second non-planar loop includes a second distal planar loop section in a second plane in the expanded configuration.

18. The catheter of claim 17, wherein the first plane intersects the second plane in the expanded configuration.

19. The catheter of claim 18, wherein the first planar loop section and second planar loop section are deformable from the expanded configuration such that the first plane lies in the second plane, the first and second planes perpendicular to the longitudinal axis.

20. The catheter of claim 18, wherein the first non-planar loop extends from a first side to a second side of a reference plane including the axis, and the second non-planar loop extends from a second side to a first side of the reference plane in the expanded configuration.
